(19) United States
(12) Patent Application Publication
(10) Pub. No.: US 2024/0138586 A1
(43) Pub. Date: May 2, 2024

(54) AUTOMATIC MATTRESS ADJUSTMENT TO IMPROVE RESTFUL SLEEP

(71) Applicant: Purple Innovation, LLC, Lehi, UT (US)

(72) Inventors: Thomas Andrew Bennett, Irvine, CA (US); James Grutta, Draper, UT (US)

(21) Appl. No.: 17/978,173

(22) Filed: Oct. 31, 2022

(51) Int. Cl. A47C 31/12; A47C 21/04; A47C 27/08; A61B 5/00; A61B 5/0205

(52) U.S. Cl. CPC A47C 31/123; A47C 21/042; A47C 21/048; A47C 27/083; A61B 5/0205; A61B 5/4836; A61B 5/6892; A61B 5/1101

(57) ABSTRACT

This disclosure relates generally to smart beds and, more specifically, to smart beds that can detect at least one component of an activity by an individual sleeping on the smart bed that is indicative of a condition that adversely affects restful sleep by the sleeping individual. Additionally, this disclosure relates to smart beds that automatically adjust a state of the sleep surface of a mattress in response to detection of the activity to reduce the activity by the sleeping individual.

### TECHNICAL FIELD

This disclosure relates generally to smart beds that automatically adjust a sleep surface of a mattress upon detecting activity on the sleep surface. More specifically, this disclosure relates to smart beds that automatically adjust a sleep surface of a mattress to reduce an activity that may indicate a condition that adversely affects restful sleep.

### SUMMARY

In various aspects, sleep sequences for smart beds are disclosed.

A smart bed of this disclosure is equipped to automatically initiate a sleep surface adjustment upon detecting an activity that may indicate a condition that adversely affects restful sleep. Such a smart bed may include a mattress that defines the sleep surface, at least one sensor associated with the sleep surface, and a processor that communicates with the at least one sensor and controls one or more functions of the mattress. A smart bed may also include a foundation for the mattress.

The mattress may have any suitable configuration. As a few examples, the mattress may comprise a conventional mattress with coils, a so-called “memory foam” mattress, a mattress with one or more bladders that may be selectively pressurized, a gel mattress, or a mattress that includes a combination of these and/or other features.

The at least one sensor may be capable of detecting motion or another activity (e.g., proximity, the application of pressure, etc.) to a surrounding area of the sleep surface of the mattress. Each sensor may comprise an accelerometer. More specifically, the at least one sensor may be positioned adjacent to the sleep surface at a location where it may detect the presence of an individual on the sleep surface. The at least one sensor may detect a respiratory rate of an individual on the sleep surface, a heart rate of an individual on the sleep surface, a temperature of at least one part of a body of an individual on the sleep surface, and/or a humidity generated by at least one part of a body of an individual on the sleep surface. The at least one sensor may even detect a position (e.g., a sleep position, etc.) of an individual on the sleep surface. In some embodiments, the at least one sensor may be positioned at a location where an individual would be expected to rest their legs when laying in a resting position or sleeping position on the sleep surface. In other embodiments, the at least one sensor may comprise a group of sensors, such as any combination of a sensor at the side of the mattress (e.g., a sensor positioned over a rail of the mattress, on a side of a rail of the mattress, within a rail of the mattress, etc.), a sensor at a location where an individual would be expected to rest their body when assuming a resting position or sleeping position on the sleep surface, and/or a sensor at a location where the individual would be expected to rest their legs when assuming a resting position or sleeping position on the sleep surface.

The processor that communicates with (e.g., receives signals from, etc.) each sensor associated with the sleep surface of the mattress may comprise one or more processing elements of a suitable type. In some embodiments, the processor may comprise one or more microcontrollers, which may execute an embedded program (e.g., firmware) to control one or more dedicated functions. The processor may be carried by the mattress (e.g., internally, adjacent to a foot of the mattress; etc.).

Based on data generated by the at least one sensor, the processor may be programmed to detect at least one component of an activity by the sleeping individual indicative of a condition that adversely affects restful sleep by the sleeping individual. The processor may be programmed to determine one or more of a respiratory rate of an individual on the sleep surface, a heart rate of an individual on the sleep surface, a temperature of a part of a body of an individual on the sleep surface, a humidity generated by a part of a body of an individual on the sleep surface, or another potential component of an activity by the sleeping individual indicative of a condition that adversely affects restful sleep by the sleeping individual. Programming of the processor may even enable it to identify the individual who is present on the sleep surface; a processor programmed in this manner may initiate the at least one function in a manner that corresponds to an identity of the individual present on the sleep surface.

In some embodiments, a determination of whether there is an activity by the sleeping individual indicative of a condition that adversely affects restful sleep by the sleeping individual may be based simply on a detected heart rate, a detected respiration, a detected temperature, detected movement of the individual or other activity.

Once the processor determines there is at least one component of an activity by the sleeping individual indicative of a condition that adversely affects restful sleep by the sleeping individual, the processor may adjust a state of the sleep surface via one or more adjustors (e.g., features of the mattress, features of the smart bed etc.). Adjusting the state of the sleep surface may include adjusting a firmness (or softness) of the mattress or a portion thereof (e.g., pressurization or depressurization of a bladder within the mattress, etc.), ventilating the mattress or a portion thereof, adjusting temperature of the mattress or a portion thereof, actuating an adjustable foundation of the mattress (e.g., to adjust an orientation of the mattress or a portion thereof, etc.), etc. Adjusting the state of the sleep surface may be customized to the individual who has been identified by the processor. A few nonlimiting examples of adjustments that may be executed include adjusting an orientation of the mattress or a portion thereof, adjusting a firmness (or softness) of the mattress or a portion thereof (e.g., pressurization of a bladder within the mattress, etc.), ventilating the mattress or a portion thereof, adjusting a temperature of the mattress or a portion thereof, actuating an anti-snore feature of the smart bed (e.g., its foundation, its mattress, etc.), otherwise affecting an individual's respiration (e.g., to a normalized, restful respiratory rate, etc.), etc.

The settings for one or more adjustments to be made to the smart bed to reduce the activity indicative of a condition that adversely affects restful sleep, may be stored. For example, the settings may be stored by memory of the smart bed. The settings stored by memory of the smart bed may be accessed by the processor of the smart bed, which may, if necessary, adjust a state of the sleep surface of the smart bed in response to detecting the activity indicative of a condition that adversely affects restful sleep.

In addition to the mattress, the at least one sensor, and the processor, a smart bed of this disclosure may include a transceiver, along with an associated antenna. The transceiver may be associated with the processor to enable the processor to communicate with a processor of a portable electronic device (e.g., a smart phone, a tablet computer, a smart watch, etc.) through a transceiver and associated antenna of the portable electronic device. The processor of the portable electronic device may execute a smart bed control app, which may enable an individual to use the portable electronic device to control operation of the smart bed and/or devices in the environment in which the smart bed is located. The smart bed control app may initiate communication between the processor of the portable electronic device and the processor of the smart bed. Alternatively, the processor of the smart bed may be programmed to wake the portable electronic device as the smart bed (e.g., a sensor, etc., of the smart bed) detects the presence of the portable electronic device or the individual in close proximity to the mattress and/or detects the presence of an individual on the sleep surface of the mattress. The processor of the smart bed may then communicate with the processor of the portable electronic device in a manner that causes the processor of the portable electronic device to initiate execution of the smart bed control app.

In embodiments where the smart bed includes a foundation, the foundation may comprise an adjustable platform. The adjustable platform may include a plurality of support surfaces, at least one motor, and a processor. The plurality of support surfaces can carry the mattress of the smart bed. The at least one motor, under control of the processor, may move the plurality of support surfaces to desired orientations, which may move a mattress carried by the plurality of support surfaces to a desired arrangement. In addition, the adjustable platform may include a transceiver, along with an associated antenna. The transceiver may establish communication with the processor of the remainder of the smart bed (e.g., a processor carried by the mattress, etc.) through the transceiver of the smart bed and/or with a processor of a portable electronic device through the transceiver of the portable electronic device. Such an arrangement may enable the smart bed control app to place the adjustable platform and a mattress carried by the adjustable platform into a desired sleeping arrangement when an individual assumes a resting position or sleeping position on the sleep surface of the mattress.

In a method of this disclosure, a smart bed initiates an adjustment of the state of the sleep surface of the mattress in response to detecting at least one component of an activity by a sleeping individual indicative of a condition that adversely affects restful sleep. Such a method may include monitoring the sleeping individual, detecting at least one component of an activity by the sleeping individual indicative of a condition that adversely affects restful sleep by the sleeping individual, and adjusting a state of the sleep surface of the mattress in response to the activity to reduce the activity by the sleeping individual.

In some embodiments, in addition to monitoring an individual on the sleep surface of the mattress and detecting at least one component of an activity by the sleeping individual indicative of a condition that adversely affects restful sleep by the sleeping individual, the method may include identifying the individual present on the sleep surface. In such embodiments, adjusting the state of the sleep surface may comprise adjusting the state of the sleep surface in a manner that corresponds to the identity of the individual present on the sleep surface.

Such a method may include establishing communication between a processor of the smart bed and a processor of a portable electronic device (e.g., a smart phone, a tablet computer, a smart watch, etc.), which may execute a smart bed control app. In embodiments where the method also includes identifying the individual present on the sleep surface of the mattress, communication may be established between the processor of the smart bed and the portable electronic device of the individual present on the sleep surface. Communication between the processor of the smart bed and the portable electronic device may occur automatically upon detecting the presence of the individual on the sleep surface, for example, in a sleep position on the sleep surface. The app may enable an individual to select one or more functions to be executed to adjust the state of the sleep surface, or to edit one or more functions of a preprogrammed sequence to adjust the state of the sleep surface.

Adjusting the state of the sleep surface of the mattress may include initiating one or more functions of the smart bed, its mattress, or the sleep surface of the mattress. Such functions may include, but are not limited to, heating and/or cooling the sleep surface to a pre-set temperature, ventilating at least a portion of the sleep surface, providing at least part of the mattress with a pre-set firmness, actuating an adjustable platform of the foundation (e.g., to adjust an orientation of at least a portion of the sleep surface, etc.), etc. Other functions of a smart bed may also be initiated. In addition to adjusting the state of the sleep surface of the mattress of the smart bed, at least one function may be initiated in an environment in which the smart bed is located. Such a function may include adjusting a temperature within the environment, playing audio (e.g., music, audiobooks, sounds conducive to rest and/or sleep, subliminal sessions to be played during sleep, white noise, etc.).

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 provides a representation of an embodiment of a smart bed according to this disclosure;

FIG. 2 provides a top view of the embodiment of the smart bed shown in FIG. 1;

FIG. 3 provides a cross-sectional representation of the embodiment of the smart bed shown in FIG. 1;

FIG. 4 provides a side view of an embodiment of an adjustable base of the embodiment of the smart bed shown in FIG. 1;

FIG. 5 is a flow diagram illustrating various acts that may occur in an embodiment of a method for adjusting the sleep surface to reduce an activity by the sleeping individual that adversely affects restful sleep;

FIG. 6 is a flow diagram illustrating another embodiment of a method for adjusting the sleep surface to reduce an activity by the sleeping individual that adversely affects restful sleep of an embodiment of a smart bed;

FIG. 7 is a flow diagram illustrating another embodiment of a method for adjusting the sleep surface to reduce an activity by the sleeping individual that adversely affects restful sleep of an embodiment of a smart bed;

FIG. 8 is a flow diagram illustrating another embodiment of a method for adjusting the sleep surface to reduce an activity by the sleeping individual that adversely affects restful sleep of an embodiment of a smart bed; and

FIG. 9 is a flow diagram illustrating another embodiment of a method for adjusting the sleep surface to reduce an activity by the sleeping individual that adversely affects restful sleep of an embodiment of a smart bed.

### DETAILED DESCRIPTION

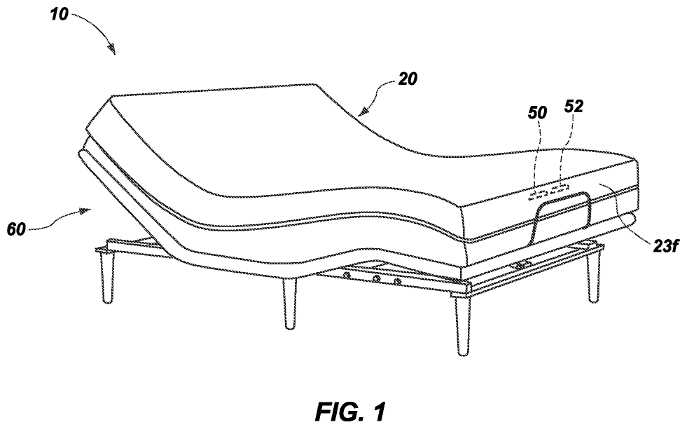

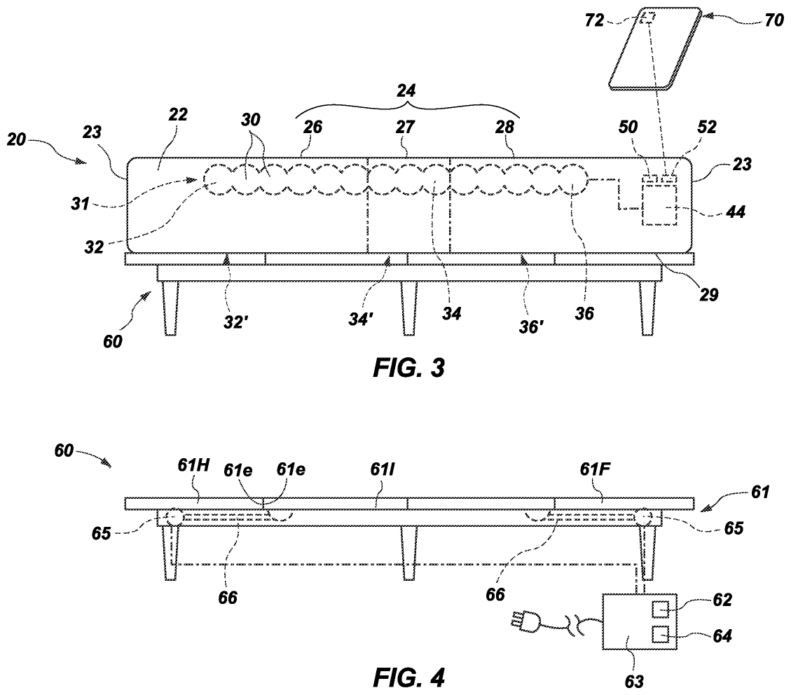

FIGS. 1-4 illustrate an embodiment of a smart bed 10. The smart bed 10 includes a mattress 20, at least one sensor 40, a processor 50, and at least one transceiver 52 and an associated antenna (not shown). In addition, the smart bed 10 may include a foundation 60. In some configurations, a single sensor 40 is used, and in other configurations, two, three, four, or more sensors 40 are used.

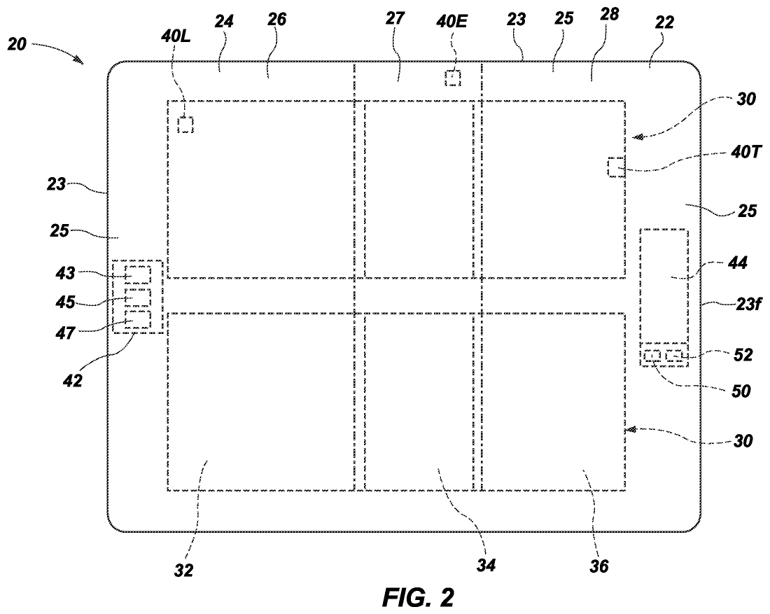

The mattress 20 of the smart bed 10 may have any suitable shape and dimensions. The mattress may include edges 23, a top surface, or sleep surface 24, between top ends of the edges 23, and a bottom surface or base 29, between bottom edges of the edges 23. The sleep surface 24 of the mattress 20 may be configured to be slept on by an individual. The sleep surface 24 may include edges 25 at locations adjacent to side edges 23 of the mattress 20, a head portion 26, an intermediate portion 27, and a foot portion 28 (FIG. 2).

Internally, the mattress 20 may include one or more pressurizable layers 31 comprised of at least one air bladder 30. In some configurations, each pressurizable layer 31 may include pressurizable sections 32, 34, 36 comprised of air bladders. The pressurizable sections 32, 34, and 36 may be pressurized independently of one another. Each pressurizable section 32, 34, 36 of the pressurizable layer 31 may be located within a corresponding section 32′, 34′, 36′ of the mattress 20 and, thus, render the corresponding sections 32′, 34′, 36′ of the mattress 20 independently pressurizable from each other. U.S. patent application Ser. No. 17/682,649, the entire disclosure of which is hereby incorporated herein, discloses a type of pressurizable layer 31 that may be included in the mattress 20.

The interior(s) of the air bladder(s) 30 may be in fluid communication with a pump 44 to pressurize the air bladder(s) 30. In some configurations, the air bladders 30 are inflatable independently from one another. Embodiments of a pressurization system, which may facilitate adjustment of a pressure (e.g., air pressure, etc.) of one or more air bladders 30, are disclosed by U.S. Pat. No. 11,213,139 and U.S. Patent Application Publication US 2022/0273113 A1, the entire disclosures of which are hereby incorporated herein.

The layers of the mattress 20 may include an extra space may define a receptacle to receive the pump 44 and any associated equipment (e.g., one or more pumps, controls, hoses, corresponding supports, an overlying cushioning element, etc.). Thus, the pump 44 and associated controls (e.g., an air pump, a ventilation pump, etc.), may reside within the body of the mattress 20. In the embodiment illustrated in FIG. 2, the receptacle may extend along a foot portion 28 of the smart bed 10 to hold the pump 44 in the foot end of the mattress 20 of the smart bed 10. In other embodiments, the pump 44 is located externally or outside the mattress 20.

The mattress 20 may comprise any suitable type of mattress, such as a conventional mattress with springs and foam, a foam mattress, a memory foam mattress, a mattress with pressurizable bladders 30 (e.g., air bladders/chambers, etc.), a mattress that includes a gel grid (e.g., those available from Purple Innovation, LLC, of Lehi, Utah and disclosed by U.S. Pat. Nos. 7,060,213, 7,076,822, and 8,919,750, which may be formed from any suitable material, including, but not limited to, an extended A-B-A triblock copolymer, such as those disclosed by U.S. Pat. Nos. 6,413,458, 6,797,765 and 7,964,664; the entire disclosures of each of the foregoing patents are hereby incorporated herein), or a mattress that includes any combination of these and/or other features.

The smart bed 10 includes at least one sensor 40E, 40L, 40T (each of which may also be referred to as a “sensor 40.” The at least one sensor 40 may be carried by the mattress 20. Without limitation, sensor(s) 40 may be positioned adjacent to the sleep surface 24 of the mattress 20. For example, a sensor 40 may be positioned within a cover of the mattress 20 in a manner that enables the sensor 40 to detect activity occurring on a sleep surface 24 of the mattress. More specifically, each sensor 40 may be positioned within a recess or a receptacle that opens to and communicates with an outer surface of a component of the mattress 20 that carries the sensor 40, with an optional cover (not shown) being placed over the recess or receptacle. In other embodiments, the sensor 40 may be incorporated into a cover or positioned between the cover and internal components (e.g., a top layer of foam, gel, etc.) of the mattress 20.

In some embodiments, the mattress 20 may carry a plurality of sensors 40. Without limitation, at least one thoracic sensor 40T may be positioned at the head portion 26 and/or the intermediate portion 27 of the mattress 20. At least one leg sensor 40L may be positioned at the foot portion 28 of the mattress 20. At least one ambient environment sensor 40E may be positioned at an edge 23 (e.g., on, adjacent to, or within a rail, etc.) of the mattress 20. The sensor(s) 40 may be used to monitor the presence of an individual on the sleep surface, the movement of at least one body part of the sleeping individual on the sleep surface 24, the temperature of at least one body part of the sleeping individual, and/or one or more vital signs of the sleeping individual (heart rate, respiration, etc.).

In a specific embodiment, the sensor(s) 40 may include an inclinometer (e.g., an inclinometer available from STMicroelectronics of Plan-les-Ouates, Switzerland, etc.), an accelerometer (e.g., an accelerometer available from MEMSIC Inc. of Andover, Massachusetts, etc.), and/or a plurality of temperature/humidity sensors (e.g., a temperature and/or humidity sensor available from Sensiron AG of Stäfa, Switzerland, etc.). Any other type of sensor may also be used as a sensor 40 (e.g., a proximity sensor, a pressure sensor, etc.)

In the illustrated embodiment of mattress 20, thoracic sensor 40T and leg sensor 40L are shown at a location where an individual is expected to lie on the sleep surface 24 of the mattress 20 as s/he sleeps, with the mattress 20 having a size that accommodates two individuals. In other embodiments, including small mattresses (e.g., twin size, twin XL (extra long) size, full size, etc.) and large mattresses (e.g., queen size, king size, etc.), thoracic sensors 40T and leg sensors 40L may be positioned along a midline of the mattress—alone or in combination with other sensor placements.

The smart bed 10 may also include a temperature control system 42. The temperature control system 42 may be any suitable type, and in some configurations the temperature control system 42 includes one or more of a ventilation system 43, a heating element 45, and/or a cooling effect 47. Embodiments of a ventilation system 43, which may ventilate part or all of the mattress 20, are disclosed by U.S. Pat. No. 11,311,111, the entire disclosure of which is hereby incorporated herein.

The processor 50 may comprise one or more processing elements of a suitable type. In some embodiments, the processor 50 may comprise one or more microcontrollers, which may execute embedded program(s) (e.g., firmware) to control one or more dedicated functions. The processor 50 may be carried by the mattress 20 of the smart bed 10 (e.g., internally, adjacent to a foot edge 23f of the mattress 20; etc.), by the foundation 60 of the smart bed 10, or elsewhere.

Activity detected by the sensor(s) 40 may be communicated to the processor 50. Similarly, sensor(s) 40 positioned in the intermediate portion 27 and/or the foot portion 28 of the mattress 20 may facilitate the detection of the presence (or absence) of an individual on the sleep surface 24 of the mattress 20. In addition, the sensor(s) 40 may be used to monitor the individual's movement (e.g., motion, position, etc.), respiration, and/or heart rate, as well as variations in respiration (e.g., snoring, variations in respiratory rate, etc.) and/or heart rate variability. The sensor(s) 40 may monitor the temperature of the individual's legs and, optionally, humidity.

The smart bed 10 also includes one or more adjustors to adjust the state of the sleep surface 24 of the mattress 20. Adjustors include, but are not limited to, the air bladder(s) 30 of a pressurizable layer 31 and associated pump(s) 44, a temperature control system 42, the foundation 60, etc. In one configuration, the state of the sleep surface 24 can be adjusted by one or more air bladders 30. The air bladder(s) 30 can be inflatable independently from one another, with at least one pump 44 in communication with interiors of the air bladders. The processor 50 can be programmed to cause the at least one pump 44 to adjust a pressure in the air bladder(s) 30 to adjust the state of the sleep surface 24.

Activity detected by the sensor(s) 40 may be communicated to the processor 50. The processor 50 may execute a program (e.g., a smart bed control app, etc.) or a part or module of the program to determine whether the data from the sensor(s) 40 indicates that there is activity by an individual on the sleep surface that is indicative of a condition that adversely affects restful sleep (e.g., snoring, variations in respiratory rate, variations in heart rate, restless leg syndrome, non-ideal temperature, etc.). If the processor 50 determines that there is activity that is indicative of a condition that adversely affects restful sleep, then the processor 50 may execute one or more programs or parts or modules thereof that affect a state of the mattress 20, the smart bed 10 of which the mattress 20 is a part, and/or the environment in which the smart bed 10 is located. This process is described in greater detail hereinafter.

In addition to the mattress 20, the sensor(s) 40, the adjustor(s), and the processor 50, a smart bed 10 of this disclosure may include a transceiver 52 and an associated antenna. The transceiver 52 may be associated with the processor 50 in a manner that enables the processor 50 to communicate with a processor 72 of a portable electronic device 70 (e.g., a smart phone, a tablet computer, a smart watch, etc.) or another device by way of a transceiver 74 of the portable electronic device 70 of other device through the antennas associated with the transceivers 52 and 74. Without limitation, the transceivers 52 and 74 may comprise one or more of a WiFi transceiver, a Bluetooth® transceiver, a near-field communication (NFC) (e.g., radiofrequency (RF), etc.) transceiver, a 2.4 GHz RF transceiver, or the like, along with associated antennas. The transceiver 52 may be carried by the mattress 20 of the smart bed 10, by the foundation 60 of the smart bed 10, or elsewhere. In some embodiments, the transceiver 52 may be associated with (e.g., located adjacent to, packaged with, etc.) the processor 50 of the smart bed 10.

A processor 72 of the portable electronic device 70 may execute a a program (e.g., the smart bed control app, etc.), which may enable an individual to use the portable electronic device 70 to control operation of the smart bed 10 and/or devices in the environment E in which the smart bed 10 is located. The individual may also be able to control specific functions of the program run by the processor 50 of the smart bed 10, such as to select and pre-program actions of the adjustor in response to detecting at least one component of an activity by the sleeping individual indicative of a condition that adversely affects restful sleep by the sleeping individual.

The smart bed control app may initiate communication between the processor 72 of the portable electronic device 70 and the processor 50 of the smart bed 10 (by way of a transceiver 74 of the portable electronic device 70 and the transceiver 52 of the smart bed 10). Alternatively, the processor 50 of the smart bed 10 may be programmed to wake the processor 72 of the portable electronic device 70 as a component of the smart bed 10 (e.g., at least one sensor 40, etc.) detects the presence of the portable electronic device 70 or the individual in close proximity to the mattress 20 and/or detects the presence of an individual on the sleep surface 24 of the mattress 20. The processor 50 of the smart bed 10 may then cause the processor 72 of the portable electronic device 70 to initiate execution of the smart bed control app.

The foundation 60 of the smart bed 10 may include any suitable foundation for the mattress 20. In some embodiments, the foundation 60 may comprise an adjustable platform (e.g., the Purple® Ascent Base′ adjustable platform, available from Purple Innovation, LLC, of Lehi, Utah, etc.). Such a foundation 60 may include a processor 62 and/or other electronics 63 associated with the processor 62. The processor 62 may control movement of one or more adjustable sections 61H, 61I, 61F of a platform 61 that carries the mattress 20. In addition, such a foundation 60 may include a transceiver 64 (e.g., a WiFi transceiver, a Bluetooth® transceiver, a near-field communication (NFC) (e.g., radiofrequency (RF), etc.) transceiver, a 2.4 GHz RF transceiver, etc.) and an associated antenna (not shown). The transceiver 64 may receive signals from the processor 50 of the smart bed 10 (by way of the transceiver 52) and/or a processor 72 of the portable electronic device 70 (by way of the transceiver 74 of the portable electronic device 70) that may cause the processor 62 of the foundation 60 to move one or more sections 61A, 61I, 61F of the platform 61 of the foundation 60 in a desired manner (e.g., to move the mattress 20 or a portion thereof to a desired orientation, to operate an anti-snore feature of the foundation 60, to move the mattress in a manner (e.g., cyclically, etc.) that will stabilize an individual's breathing at a certain rate, etc.). In other embodiments, the foundation 60 may comprise a stationary foundation (e.g., a platform style bed frame, a conventional bed frame and box spring, etc.).

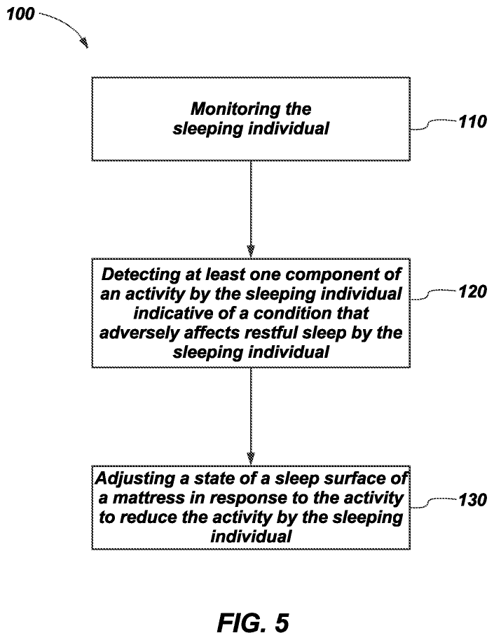

FIG. 5 is a diagram illustrating the flow of an embodiment of a method 100 of initiating an adjustment of the smart bed 10, such as a smart bed depicted by FIGS. 1-4. The method 100 may include monitoring the sleeping individual in a resting position or a sleeping position on the sleep surface 24 (FIGS. 1-4) of a mattress 20 (FIGS. 1-4) of the smart bed 10 (reference 110), detecting at least one component of an activity by the sleeping individual indicative of a condition that adversely affects restful sleep by the sleeping individual (reference 120), and adjusting a state of a sleep surface of a mattress in response to the activity to reduce the activity by the sleeping individual (reference 130).

Monitoring the sleeping individual may include monitoring one or more of: breathing/respiration of the sleeping individual, a heart rate of the sleeping individual, movement of at least one body part of the sleeping individual, temperature of at least one body part of the sleeping individual, and/or the position of bed linens relative to a position of the sleeping individual on the sleep surface 24 of the mattress 20. As the sleeping individual is monitored, the sensor(s) 40 can constantly or nearly constantly send data to the processor 50 of the smart bed 10.

The processor 50 may execute a program (e.g., the smart bed control app, etc.) or a part or module thereof to determine whether the data from the sensor(s) 40 indicates that there is activity by an individual on the sleep surface 24 that is indicative of a condition that adversely affects restful sleep (e.g., snoring, variations in breathing rate, variations in heart rate, restless leg syndrome, non-ideal temperature, etc.). In one embodiment, such a program can access a library of known parameters that are indicative of a condition that adversely affects restful sleep, and the program may compare the parameters received by the processor 50 from the sensor(s) 40 with known parameters indicative of a condition that adversely affects restful sleep. In another embodiment, the program can additionally or alternatively access a library of known parameters that are indicative of a condition that occurs during restful sleep (e.g., a predetermined ideal temperature or temperature range, a predetermined heart rate or range, a predetermined respiration rate or range, etc.), and the program may compare the known parameters received by the processor 50 from the sensor(s) 40 to determine if the data received by the processor 50 fall(s) within the acceptable ranges of the known parameters indicative of a condition that occurs during restful sleep.

If the processor 50 determines that there is activity that is indicative of a condition that adversely affects restful sleep, the processor 50 can cause an adjustor (e.g., the air bladder(s) 30 of a pressurizable layer 31 and associated pump(s) 44, a temperature control system 42, the foundation 60, etc.) to adjust the state of the sleep surface 24 of the mattress 20 in a manner that reduces the activity by the individual. For example, the processor 50 may execute one or more programs (e.g., the smart bed control app, etc.) or parts or modules thereof that affect a state of the sleep surface 24, the mattress 20, the smart bed 10 of which the mattress 20 is a part, and/or the environment in which the smart bed 10 is located.

The programming of the processor 50 can also include one or more machine learning algorithms to enable the processor 50 to identify patterns and make decisions with respect to identifying conditions that adversely affect an individual's restful sleep and adjusting the state of the sleep surface 24 of the mattress 20 through data received over time (i.e., experience in monitoring an individual's sleep). In systems where the processor 50 identifies the individual on the sleep surface 24, the machine learning of the processor 50 may be unique to the particular individual such that adjusting a state of a sleep surface 24 of a mattress 20 to reduce activity by the individual that is indicative of a condition that adversely affects the individual's ability to achieve restful sleep comprises adjusting the state of the sleep surface 24 in a manner that is unique to the particular individual.

With returned reference to FIG. 5, the sensor(s) 40 and processor 50 of the smart bed 10 may monitor the sleeping individual (reference 110 in FIG. 5). During monitoring, the sensor(s) 40 and processor 50 may detect at least one component of an activity by the sleeping individual indicative of a condition that adversely affects restful sleep by the sleeping individual (reference 120 of FIG. 5). Upon detecting the at least one component, the processor 50 may adjust a state of the sleep surface 24 of the mattress 20 to reduce the activity by the sleeping individual.

Adjusting the state of the sleep surface 24 of the mattress 20 to reduce the activity by the sleeping individual can include one or more of adjusting pressurization of the air bladder(s) 30 of the mattress 20, adjusting an adjustable platform 61 of the foundation 60 of the mattress 20, adjusting a temperature of the mattress 20, etc. In one embodiment, the adjustor comprises one or more air bladders 30 that are inflatable independent from one another. At least one pump 44 may be in fluid communication with the interior(s) of the air bladder(s) 30, and the processor 50 may be programmed to cause the pump 44 to repeatedly adjust a pressure in the air bladder(s) 30 to generate movement in the sleep surface 24 of the mattress 20 that reduces the activity by the sleeping individual indicative of a condition that adversely affects restful sleep.

In another embodiment, the adjustor is a platform 61 of the foundation 60, and the processor 50 may send a signal through the transceiver 52 of the smart bed to the transceiver 64 of the foundation 60 to cause the processor 62 of the foundation 60 to move one or more sections 61A, 61I, 61F of the platform 61 of the foundation 60 to adjust the mattress 20 and the sleep surface 24 thereof in a manner that moves the head and/or body of the sleeping individual to reduce the activity by the sleeping individual indicative of a condition that adversely affects restful sleep.

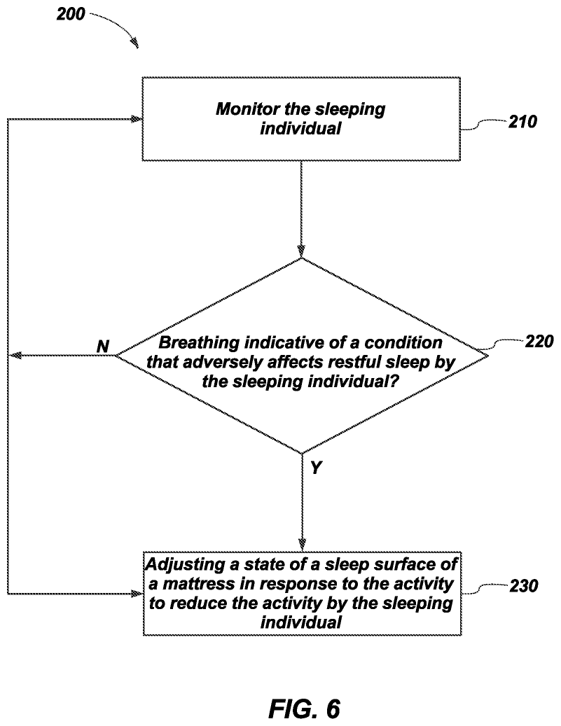

With reference to FIGS. 6-9, embodiments of method are shown that may enable the processor 50 to detect at least one component of an activity by the sleeping individual indicative of a condition that adversely affects restful sleep by the sleeping individual, and automatically adjust the sleep surface 24 of the mattress 20 in response. Monitoring the sleeping individual may include, for example, monitoring a breathing rate of the sleeping individual, a heart rate of the sleeping individual, movement of at least one body part of the sleeping individual, a temperature of at least one body part of the sleeping individual, a position of a cover relative to a position of the sleeping individual and/or the sleep surface 24, and/or other parameters indicative of the state of restful sleep of an individual.

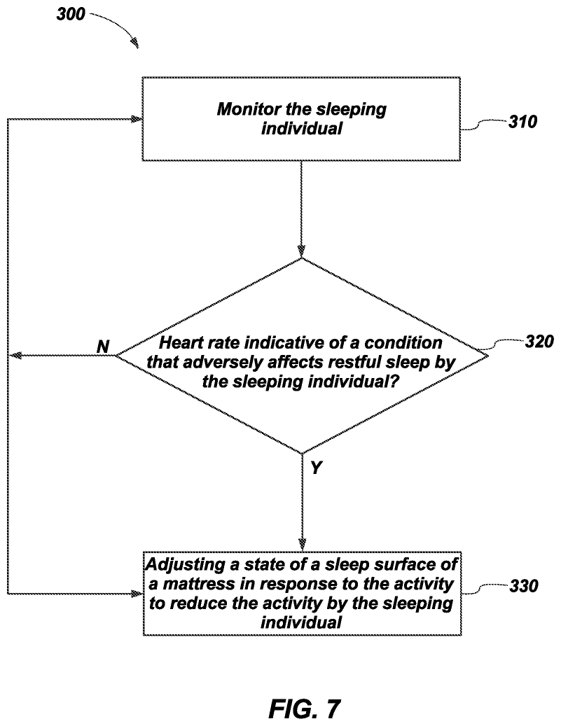

With reference to FIG. 7, a method 200 is shown for monitoring a sleeping individual on the sleep surface 24 of the mattress 20 (reference 210). The processor 50 may receive data from the sensor(s) 40 continuously or nearly continuously, and may continuously or nearly continuously query to determine whether the breathing of the individual is indicative of a condition that adversely affects restful sleep (reference 220). Several breathing patterns can be indicative of a condition that adversely affects restful sleep.

In one embodiment, the breathing indicative of a condition that adversely affects restful sleep is snoring, which may be detected with a sensor 40 that comprises an inclinometer. Upon detecting snoring by the sleeping individual, the system may adjust a state of the sleep surface 24 of the mattress 20 to reduce the snoring by the sleeping individual (reference 230). In one embodiment, the processor 50 may send a signal to the pump(s) 44 to adjust a pressure within the bladder(s) 30 and, thus, a firmness or softness of at least a portion of the sleep surface 24 in a manner that moves a head or a body of the sleeping individual (e.g., to a new position, in a continuous, wave-like fashion, etc.) to reduce the snoring. Alternatively, the processor 50 may send a signal to the processor 62 of the foundation 60 to adjust the platform 61 to move the mattress 20 and the sleep surface 24 thereof in a manner that moves the head and/or body of the sleeping individual (e.g., to a new position, in a continuous back-and-forth or rocking fashion, etc.) to reduce the snoring.

In another embodiment, the breathing that is indicative of a condition that adversely affects restful sleep is a variation, or change, in respiratory rate (e.g., an inconsistent respiratory rate, etc.), which may be detected with a sensor that comprises an inclinometer. Upon detecting a variation in respiratory rate, the system may adjust a state of the sleep surface 24 of the mattress 20 to “reset” the individual's respiratory rate. In one embodiment, the processor 50 may adjust the state of the sleep surface 24 of the mattress 20 by causing the pump(s) 44 to cyclically adjust a pressure within the bladder(s) 30 (e.g., repeatedly inflate and deflate the bladder(s) 30 or a portion thereof, etc.; e.g., all at once, in a wavelike manner, etc.) at a rate that corresponds to a restful respiratory rate for the individual, which may facilitate normalization of the individual's respiratory rate at the restful respiratory rate.

With reference to FIG. 7, the processor 50 may receive data from the sensor(s) 40 continuously or nearly continuously to monitor the sleeping individual on the sleep surface 24 of the mattress 20 ((reference 310), and may continuously or nearly continuously query to determine whether the heart rate of the individual is indicative of a condition that adversely affects restful sleep (reference 320). In one embodiment, an elevated heart rate of the individual is indicative of a condition that adversely affects restful sleep. In one embodiment, the processor 50 may adjust the state of the sleep surface 24 of the mattress 20 (reference 330) by causing the pump(s) 44 to adjust a pressure within the bladder(s) 30 and, thus, a firmness of softness of at least a portion of the sleep surface 24 in a manner that moves a head or a body of the sleeping individual (e.g., to a new position, in a continuous, wave-like fashion, etc.) to reduce their heart rate. In another embodiment, the processor 50 may send a signal to the processor of the foundation to adjust the platform to move the mattress 20 and the sleep surface 24 thereof in a manner that moves the head and/or body of the sleeping individual (e.g., to a new position, in a continuous back-and-forth or rocking fashion, etc.) to reduce the individual's heart rate. In yet other configurations, the processor 50 may cause the temperature control system 42 to ventilate or adjust the temperature of at least a portion of the sleep surface 24 to reduce the individual's heart rate.

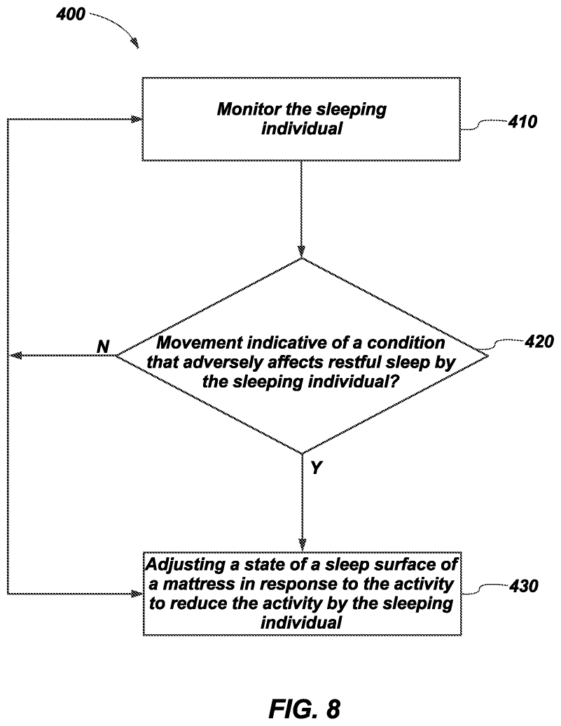

With reference to FIG. 8, the processor 50 may monitor a sleeping individual on the sleep surface 24 of the mattress 20 (reference 410) by continuously or nearly continuously receiving data from the sensor(s) 40 and by querying the data to determine whether movement of a body part of the sleeping individual is indicative of a condition that adversely affects restful sleep (reference 420). In one embodiment, the condition that adversely affects restful sleep is movement related to restless leg syndrome. Upon detecting movement indicative of restless leg syndrome by the sleeping individual, the processor 50 may send a signal to adjust a state of the sleep surface 24 of the mattress 20 to reduce the movement by the sleeping individual (reference 430). In one embodiment, the processor 50 may send a signal to the pump(s) 44 to adjust a pressure within the bladder(s) 30 and, thus, a firmness or softness of at least a portion of the sleep surface 24 of the mattress 20 in a manner that moves a head or a body of the sleeping individual to reduce the movement associated with restless leg syndrome (e.g., by moving the head or body to a new position, by moving the head or body in a constant wave-like fashion, etc.). Even more specifically, the processor 50 may send a signal to the pump(s) 44 to increase and/or decrease pressure in two or more sections 34, 36 of the bladder(s) 30. The signal from the processor 50 may cause the pump(s) 44 to repeatedly adjust a pressure in at least one air bladder 30 of the two or more air bladders to generate continuous, wave-like movement of at least part of the sleep surface 24 of the mattress 20 to reduce the leg and/or foot movement.

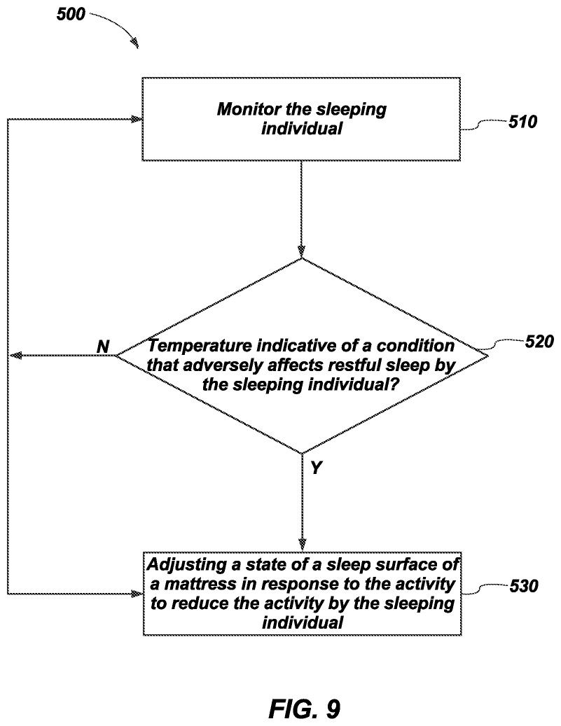

With reference to FIG. 9, the processor 50 may monitor a sleeping individual on a sleep surface 24 of a mattress 20 (reference 510) by continuously or nearly continuously receiving data from one or more sensor(s) 40, and querying data to determine whether a temperature of a body part of the sleeping individual is indicative of a condition that adversely affects restful sleep (reference 520). In one embodiment, an elevated temperature of a body part, or a temperature of a body part above an acceptable threshold, is indicative of a condition that adversely affects restful sleep is an elevated temperature. Upon detecting the temperature indicative of the condition that adversely affects restful sleep by the sleeping individual, the processor 50 may send a signal to one or more adjustors to adjust a state of the sleep surface of the mattress to reduce the temperature (reference 530). In one embodiment, the processor 50 may send a signal to a temperature control system 42. The temperature control system 42 may initiate a ventilation system 43 and/or a cooling effect 47 to adjust a state of at least a portion of the sleep surface 24.

The processor 50 may be programmed to distinguish the activity of the sleeping individual that adversely affects restful sleep from other events. Some examples of such an event include an individual merely coughing but not snoring, moving their legs to adjust their sleep position but not moving their leg/foot in a manner that indicates restless leg syndrome, etc.

In some embodiments, the processor 50 may communicate with a processor 72 executing a smart bed control app on a portable electronic device 70 (e.g., a smart phone, a tablet computer, a smart watch, etc.) through respective transceivers 52 and 74, which may send a data log to the portable electronic device 70 to inform the individual regarding detected activity by the sleeping individual indicative of a condition that adversely affects restful sleep by the sleeping individual, and specific adjustments made. In some embodiments, the portable electronic device 70 may communicate with the processor 50 to instruct the processor 50 regarding adjustments to be made to the state of the sleep surface 24 of the mattress 20. In this manner, an individual can customize what instructions the processor 50 will send automatically upon detection of particular activities.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A smart bed, comprising:
a mattress including:
a sleep surface;
at least one sensor associated with the sleep surface, the at least one sensor comprising a sensor that detects at least one component of an activity by an individual on the sleep surface indicative of a condition that adversely affects restful sleep by the individual;
an adjustor that adjusts a state of the sleep surface; and
a processor in communication with the at least one sensor and the adjustor, the processor programmed to:
process signals from the at least one sensor to detect the activity by the individual on the sleep surface indicative of the condition that adversely affects restful sleep by the individual; and
upon detecting the activity by the individual, cause the adjustor to adjust the state of the sleep surface in a manner that reduces the activity by the individual.

2. The smart bed of claim 1, wherein the at least one sensor comprises an inclinometer.

3. The smart bed of claim 2, wherein the inclinometer is positioned at a location on the sleep surface that receives a chest and/or a back of the individual.

4. The smart bed of claim 3, wherein the processor processes signals from the inclinometer to detect respiration that disrupts the individual's sleep.

5. The smart bed of claim 4, wherein:
the adjustor comprises air bladders that are inflatable independently from one another and a pump in communication with interiors of the air bladders; and
the processor is programmed to cause the pump in communication with the interiors of the air bladders to adjust a pressure in at least one air bladder of the air bladders to affect a change in the subject's respiration.

6. The smart bed of claim 1, wherein the at least one sensor comprises a pressure or motion sensor.

7. The smart bed of claim 6, wherein the sensor is positioned at a location on the sleep surface that receives legs and/or feet presence of the individual.

8. The smart bed of claim 7, wherein the processor receives signals from the at least one sensor to detect a leg and/or foot movement indicative of restless leg syndrome.

9. The smart bed of claim 8, wherein:
the adjustor comprises air bladders that are inflatable independently from one another and a pump in communication with interiors of the air bladders; and
the processor is programmed to cause the pump to repeatedly adjust a pressure in at least one air bladder of the air bladders to generate movement in the mattress that reduces the leg and/or foot movement.

10. The smart bed of claim 1, wherein the adjustor comprises:
air bladders that are inflatable independently from one another; and
a pressurization system that controls a pressure of each air bladder of the air bladders.

11. The smart bed of claim 1, wherein the adjustor comprises:
a temperature control system.

12. The smart bed of claim 11, wherein the temperature control system includes a ventilation system.

13. The smart bed of claim 11, wherein the temperature control system includes at least one heating element.

14. The smart bed of claim 11, wherein the temperature control system includes at least one cooling effect.

15. The smart bed of claim 4, wherein:
the adjustor comprises an adjustable frame that carries the mattress; and
the processor is programmed to cause the adjustable frame that carries the mattress to adjust an orientation of at least a portion of the mattress to change an orientation of a head and/or a body of the individual.

16. A method for addressing an activity by a sleeping individual indicative of a condition that adversely affects restful sleep by the sleeping individual, comprising:
monitoring the sleeping individual;
detecting at least one component of the activity by the sleeping individual; and
adjusting a state of a sleep surface of a mattress in response to the activity to reduce the activity by the sleeping individual.

17. The method of claim 16, wherein monitoring the sleeping individual comprises monitoring at least one of breathing of the sleeping individual, a heart rate of the sleeping individual, movement of at least one body part of the sleeping individual, a temperature of at least one body part of the sleeping individual, and a position of a cover relative to a position of the sleeping individual.

18. The method of claim 16, wherein detecting the at least one component of the activity by the sleeping individual comprises detecting snoring by the sleeping individual.

19. The method of claim 18, wherein adjusting the state of the sleep surface comprises adjusting a pressure of at least a portion of the sleep surface in a manner that moves a head or a body of the sleeping individual to reduce the snoring.

20. The method of claim 16, wherein detecting the at least one component of the activity by the sleeping individual comprises detecting a leg and/or foot movement indicative of restless leg syndrome.

21. The method of claim 20, wherein adjusting the state of the sleep surface comprises at least one of:
causing a portion of the sleep surface to move a leg and/or foot of the sleeping individual in a manner that reduces further movement of the leg and/or foot of the sleeping individual; and
adjusting a temperature of a portion of the sleep surface to reduce further movement of the leg and/or foot of the sleeping individual.

22. The method of claim 20, wherein detecting the at least one component of the activity by the sleeping individual comprises detecting a temperature of at least a portion of the sleeping individual.

23. The method of claim 22, wherein detecting the at least one component of the activity by the sleeping individual comprises determining whether or not at least the portion of the sleeping individual is covered by bed linens.

24. The method of claim 22, wherein adjusting the state of the sleep surface comprises adjusting a temperature of at least a portion of the sleep surface on which the portion of the sleeping individual lies.
